(19) United States
(12) Patent Application Publication
(10) Pub. No.: US 2026/0146246 A1
(43) Pub. Date: May 28, 2026

(54) Fusion Geminin Protein

(71) Applicant: Integrated DNA Technologies, Inc., Coralville, IA (US)

(72) Inventors: Vaughn THADA, Coralville, IA (US); Karthik MURUGAN, Coralville, IA (US); Garrett R. RETTIG, Coralville, IA (US)

(73) Assignee: Integrated DNA Technologies, Inc., Coralville, IA (US)

(21) Appl. No.: 19/398,353

(22) Filed: Nov. 24, 2025

Related U.S. Application Data
(60) Provisional application No. 63/725,260, filed on Nov. 26, 2024.

(51) Int. Cl. C12N 15/11; C07K 14/47; C12N 9/22; C12N 15/86; C12N 15/88

(52) U.S. Cl. CPC C12N 15/111; C07K 14/4703; C12N 9/226; C12N 15/86; C12N 15/88; C07K 2319/09; C07K 2319/40; C12N 2310/20; C12N 2750/14143; C12N 2800/22

(57) ABSTRACT

A CRISPR-Cas-Geminin system that maintains potent on-target editing activity but has reduced off-target editing activity relative to wild-type Cas, and methods of use.

This application claims the benefit of U.S. Ser. No. 63/725,260, filed Nov. 26, 2024, the entirety of which is incorporated herein by reference.

### REFERENCE TO AN ELECTRONIC SEQUENCE LISTING

The contents of the electronic sequence listing 63910010PV01_Sequence_11172024.xml; Size: 143 Kbytes; and Date of Creation: 11/17/2024 is herein incorporated by reference in its entirety.

### BACKGROUND

CRISPR-Cas9 genome editing is a method commonly employed for the disruption of gene function. Upon cellular delivery, the Cas9 nuclease is directed to a specific genomic location by a guide RNA (gRNA) in a sequence-dependent manner where it generates a double-strand break (DSB). Inaccurate repair of the DSB by non-homologous end-joining (NHEJ) can result in an insertion or deletion (indel), thereby leading to a frameshift mutation and loss of gene expression. Alternatively, the DSB can be repaired by homologous recombination (HR), which generally results in restoration of the original sequence and no loss of gene expression. HR occurs when the damaged sister chromatid uses sequence information on the undamaged sister chromatid to facilitate repair. In CRISPR genome editing experiments, HR-mediated repair using sequence information on an exogenously supplied donor template can result in donor genomic integration (knock-in). NHEJ and HR function antagonistically in cells, and as such, modulation of these pathways in CRISPR experiments alters the observed frequency of indels and donor template integration events.

Genomic integration of exogenous templates greater than 500 base-pairs using CRISPR-Cas9 is often inefficient due to the propensity of mammalian cells to repair DSBs by NHEJ rather than HR. However, knock-in rates can be increased through a multitude of mechanisms including NHEJ inhibition, regulation of origin firing, 53BP1 modulation, and/or cell cycle perturbations (1-4).

Several studies that have examined how cell cycle regulation can be exploited to increase HR-mediated knock-in of exogenous donor templates have explored the effect of fusing the N-terminal amino acids of Geminin to Cas9. Geminin is expressed in the S, G2, and M phases of the cell cycle and prevents DNA re-replication. In G1 phase, Geminin is ubiquitinated and degraded in an APC^{Cdh1}-dependent manner, and this degradation is dependent on Geminin amino acids 23-31 (5). To date, all peer-reviewed studies examining Geminin-Cas9 fusions have assessed how fusion of the first 110 amino acids of Geminin (Geminin 1-110) to Cas9 affects knock-in rates. Because, like full-length Geminin, Geminin 1-110 undergoes G1-dependent degradation (6), previous studies hypothesized that Cas9 fused to Geminin 1-110 would undergo degradation in the HR-restrictive G1 phase but be expressed in the HR-permissive S and G2 phases. As such, it was theorized that restricting Cas9 expression to HR-permissive cell cycle stages would increase HR-mediated knock-in rates. While one study demonstrated G1-dependent degradation of Cas9-Geminin 1-110 (7), that same study and others observed only a modest, or no increase in exogenous donor knock-in rates (7-10).

In order to improve the Cas9-Geminin system to achieve higher knock-in rates, the inventors engineered plasmids where various modified Geminin constructs having the amino acids 2-110, 2-60, 2-40, or 18-36 of Geminin (numbered from the first amino acid of N-terminus of Geminin) were fused to the C-terminus of HiFi Cas9. HiFi Cas9-Geminin mRNA was synthesized and delivered to cells, and knock-in percentages of long (>500 base pairs) double-stranded DNA (dsDNA) donor templates were assessed. Minimal increases in knock-in rates were observed, but surprisingly, the fusion of Geminin amino acids 18-36 to the C-terminus of HiFi Cas9 or WT Cas9 caused increased indels in cells when delivered as mRNA.

There exists a need to provide a CRISPR-Cas9-Geminin system that maintains potent on-target editing activity but has reduced off-target editing activity. The present disclosure provides compositions and methods for an enhanced Cas9-Geminin system, which generates higher indel percentages at on-target sites compared to that of a non-fusion Cas9 while generating similar indel levels at off-target sites.

All references cited herein are incorporated herein by reference in their entireties.

### BRIEF SUMMARY

Various embodiments contemplated herein may include, but need not be limited to, one or more of the following: Embodiment 1. An engineered, non-naturally occurring Clustered Regularly Interspaced Short Palindromic Repeats (CRISPR)-Cas-Geminin system comprising: a) a first regulatory element operable in a eukaryotic cell operably linked to at least one nucleotide sequence encoding a CRISPR-Cas system guide RNA (“gRNA”) that hybridizes with a target sequence; b) a second regulatory element operable in a eukaryotic cell operably linked to a nucleotide sequence encoding a fusion protein comprising Geminin sequence amino acids fused to the C-terminus of a Cas protein, thereby forming a Cas-Geminin fusion protein; c) wherein components (a) and (b) are located on same or different vectors of the system, wherein the gRNA targets and hybridizes with the target sequence and the Cas-Geminin fusion protein cleaves the DNA molecule, wherein the Cas-Geminin fusion protein and the gRNA do not naturally occur together. Embodiment 2. The system of embodiment 1, wherein the Cas-Geminin fusion protein comprises a Cas protein selected from the group consisting of wild-type Cas9, SaCas9, NmCas9, St1Cas9, HiFi-Cas9, wild-type Cas12, Cas12a, Cas12f, Cas13, Cas3, Caf1, AsCas12a, LbCas12a, and MAD7. Embodiment 3. The system of any one of embodiments 1-2, wherein the Cas-Geminin fusion protein comprises at least one Geminin variant comprising amino acids 2-60, 2-50, 2-40, 5-60, 5-50, 5-40, 10-60, 10-50, 10-40, 15-60, 15-50, 15-40, 18-60, 18-50, 18-40, and 18-36 of Geminin, wherein the amino acids are numbered from the first amino acid of the N-terminus of Geminin that is fused to the C-terminus of the Cas protein, thereby forming the Cas-Geminin fusion protein. Embodiment 4. The system of any one of embodiments 1-3 wherein the active CRISPR-Cas-Geminin fusion protein displays reduced off-target editing activity and maintains on-target editing activity relative to a wild-type CRISPR/Cas endonuclease system having the wild-type Cas9 protein of SEQ ID NO: 5. Embodiment 5. The system of any one of embodiments 1-4 wherein the target sequence is in a DNA molecule in a eukaryotic cell, wherein the DNA molecule encodes, and the eukaryotic cell expresses, at least one gene product. Embodiment 6. The system of any one of embodiments 1-5 wherein the CRISPR-Cas-Geminin fusion protein further comprises one or more Nuclear Localization Signals (NLS). Embodiment 7. The system of any one of embodiments 1-6 wherein the CRISPR-Cas-Geminin fusion protein further comprises a tag selected from the group consisting of an HA tag, an AviTag, a Calmodulin-tag, a polyglutamate tag, an E-tag, and a FLAG-tag. Embodiment 8. The system of any one of embodiments 1-7 wherein the CRISPR-Cas-Geminin fusion protein further comprises a linker sequence, optionally wherein the linker sequence has the amino acid sequence GGGGS (SEQ ID NO: 22). Embodiment 9. The system of any one of embodiments 1-8 wherein the system alters expression of at least one gene product. Embodiment 10. The system of any one of embodiments 1-9 wherein the Cas-Geminin fusion protein is codon optimized for expression in the eukaryotic cell. Embodiment 11. The system of any one of embodiments 1-10 wherein the eukaryotic cell is a human cell. Embodiment 12. The system of any one of embodiments 1-11 wherein the system increases expression of one or more gene products. Embodiment 13. The system of any one of embodiments 1-12 wherein the system decreases expression of one or more gene products. Embodiment 14. The system of any one of embodiments 1-13 wherein the CRISPR-Cas is encoded by a nucleotide sequence of SEQ ID NO: 3. Embodiment 15. The system of any one of embodiments 1-14 wherein the CRISPR-Cas has the amino acid sequence of SEQ ID NO: 26. Embodiment 16. The system of any one of embodiments 1-15 wherein the Geminin is Geminin 18-36 which is encoded by a nucleotide sequence of SEQ ID NO: 6. Embodiment 17. The system of any one of embodiments 1-16 wherein the Geminin is Geminin 18-36 which has the amino acid sequence of SEQ ID NO: 36. Embodiment 18. The system of any one of embodiments 1-17 wherein the Cas-Geminin fusion protein is Cas-Geminin 18-36 which is encoded by a nucleotide sequence of SEQ ID NO: 4. Embodiment 19. The system of any one of embodiments 1-18 wherein the Cas-Geminin fusion protein is Cas-Geminin 18-36 which has the amino acid sequence of SEQ ID NO: 29.

Embodiment 20. A method of performing gene editing having reduced off-target editing activity and/or increased on-target editing activity, comprising: contacting a candidate editing target site locus with an active CRISPR-Cas-Geminin system comprising: a) a first regulatory element operable in a eukaryotic cell operably linked to at least one nucleotide sequence encoding a CRISPR-Cas system guide RNA (“gRNA”) that hybridizes with a target sequence; b) a second regulatory element operable in a eukaryotic cell operably linked to a nucleotide sequence encoding a fusion protein comprising Geminin sequence amino acids fused to the C-terminus of Cas protein, thereby forming a Cas-Geminin fusion protein; c) wherein components (a) and (b) are located on same or different vectors of the system, wherein the gRNA targets and hybridizes with the target sequence and the Cas-Geminin fusion protein cleaves the DNA molecule wherein the Cas-Geminin fusion protein and the gRNA do not naturally occur together. Embodiment 21. The method of embodiment 20, wherein the Cas-Geminin fusion protein comprises a Cas protein selected from the group consisting of wild-type Cas9, SaCas9, NmCas9, St1Cas9, HiFi-Cas9, wild-type Cas12, Cas12a, Cas12f, Cas13, Cas3, Caf1, AsCas12a, LbCas12a, and MAD7. Embodiment 22. The method of any one of embodiments 20-21 wherein the Cas-Geminin fusion protein comprises at least one Geminin variant comprising amino acids 2-60, 2-50, 2-40, 5-60, 5-50, 5-40, 10-60, 10-50, 10-40, 15-60, 15-50, 15-40, 18-60, 18-50, 18-40, and 18-36 of Geminin, wherein the amino acids are numbered from the first amino acid of the N-terminus of Geminin that is fused to the C-terminus of the Cas protein, thereby forming a Cas-Geminin fusion protein. Embodiment 23. The method of any one of embodiments 20-22 wherein the active CRISPR-Cas-Geminin fusion protein displays reduced off-target editing activity and maintained on-target editing activity relative to a wild-type CRISPR/Cas endonuclease system having the wild-type Cas9 protein of SEQ ID NO: 1. Embodiment 24. The method of any one of embodiments 20-23 wherein the target sequence is in a DNA molecule in a eukaryotic cell, wherein the DNA molecule encodes, and the eukaryotic cell expresses, at least one gene product Embodiment 25. The method of any one of embodiments 20-24 wherein the CRISPR-Cas-Geminin fusion protein further comprises one or more Nuclear Localization Signals (NLS). Embodiment 26. The method of any one of embodiments 20-25 wherein the CRISPR-Cas-Geminin fusion protein further comprises a tag selected from the group consisting of an HA tag, an AviTag, a Calmodulin-tag, a polyglutamate tag, an E-tag, and a FLAG-tag. Embodiment 27. The method of any one of embodiments 20-26 wherein the CRISPR-Cas-Geminin fusion protein further comprises a linker sequence, optionally wherein the linker sequence has the amino acid sequence GGGGS (SEQ ID NO: 22). Embodiment 28. The method of any one of embodiments 20-27 wherein expression of at least one gene product is altered. Embodiment 29. The method of any one of embodiments 20 28 wherein the Cas-Geminin fusion protein is codon optimized for expression in the eukaryotic cell. Embodiment 30. The method of any one of embodiments 20-29 wherein the eukaryotic cell is a human cell. Embodiment 31. The method of any one of embodiments 20-30 wherein the expression of one or more gene products is increased. Embodiment 32. The method of any one of embodiments 20-31 wherein the expression of one or more gene products is decreased. Embodiment 33. The method of any one of embodiments 20-32 wherein the CRISPR-Cas is encoded by a nucleotide sequence of SEQ ID NO: 3. Embodiment 34. The method of any one of embodiments 20-33 wherein the CRISPR-Cas has the amino acid sequence of SEQ ID NO: 26. Embodiment 35. The method of any one of embodiments 20-34 wherein the Geminin 18-36 is encoded by a nucleotide sequence of SEQ ID NO: 6. Embodiment 36. The method of any one of embodiments 20-35 wherein the Geminin 18-36 has the amino acid sequence of SEQ ID NO: 36. Embodiment 37. The method of any one of embodiments 20-36 wherein the CRISPR-Cas-Geminin 18-36 is encoded by a nucleotide sequence of SEQ ID NO: 4. Embodiment 38. The method of any one of embodiments 20-37 wherein the CRISPR-Cas-Geminin 18-36 has the amino acid sequence of SEQ ID NO: 29.

Embodiment 39. A method of delivering the CRISPR system of any one of embodiments 1-19 to a mammalian cell, the method comprising the steps of: (a) providing a first viral vector component encoding a CRISPR-Cas system guide RNA (“gRNA”) that hybridizes with a target sequence; (b) providing a second viral vector component encoding a fusion protein comprising Geminin sequence amino acids fused to the C-terminus of Cas protein, thereby forming a Cas-Geminin fusion protein; wherein components (a) and (b) are located on same or different vectors of the system; and (c) transducing the mammalian cell with the viral vector(s) under conditions sufficient to express the Cas-Geminin fusion protein and the gRNA, wherein the Cas-Geminin fusion protein and the gRNA form a complex that binds to and edits the target sequence. Embodiment 40. A method for delivering the CRISPR system of any one of embodiments 1-19 to a mammalian cell, comprising: (a) providing lipid nanoparticles encapsulating a CRISPR-Cas system guide RNA (“gRNA”) that hybridizes with a target sequence; (b) providing lipid nanoparticles encapsulating an mRNA encoding a fusion protein comprising Geminin sequence amino acids fused to the C-terminus of Cas protein, thereby forming a Cas-Geminin fusion protein; wherein components (a) and (b) are located on same or different vectors of the system, (c) introduction of the mammalian cell with lipid nanoparticles under conditions sufficient to express the Cas-Geminin fusion protein and the gRNA, wherein the Cas-Geminin fusion protein and guide RNA are expressed and form a complex to edit a specific target sequence in the cell's genome.

Embodiment 41.A method of performing gene editing having reduced off-target editing activity and/or increased on-target editing activity, comprising: (a) providing a ribonucleoprotein (RNP) complex comprising the CRISPR system of any one of embodiments 1-19; and (b) introducing the RNP complex into the mammalian cell using electroporation, wherein the CRISPR system binds to and edits a target sequence within the genome of the mammalian cell.

Embodiment 42. A method of delivering the CRISPR system of any one of embodiments 1-19 to a mammalian cell, comprising: (a) preparing a lipofection reagent comprising a first regulatory element operable in a eukaryotic cell operably linked to at least one nucleotide sequence encoding a CRISPR-Cas system guide RNA (“gRNA”) that hybridizes with a target sequence; (b) a second regulatory element operable in a eukaryotic cell operably linked to a nucleotide sequence encoding a fusion protein comprising Geminin sequence amino acids fused to the C-terminus of Cas protein, thereby forming a Cas-Geminin fusion protein; wherein components (a) and (b) are located on the same or different vectors of the system, and (c) applying the lipofection reagent to the mammalian cell, wherein the Cas-Geminin fusion protein and guide RNA are expressed and form a complex to edit a specific target sequence in the cell's genome.

Embodiment 43. A method of targeted delivery of the CRISPR system of any one of embodiments 1-19 to a mammalian cell, comprising: (a) preparing exosomes encapsulating a first nucleotide sequence encoding a CRISPR-Cas system guide RNA (“gRNA”) that hybridizes with a target sequence; and (b) preparing exosomes encapsulating a second nucleotide sequence encoding a fusion protein comprising Geminin sequence amino acids fused to the C-terminus of Cas protein, thereby forming a Cas-Geminin fusion protein; wherein components (a) and (b) are located on same or different vectors of the system; and (c) delivering the engineered exosomes to the mammalian cell under conditions that allow the Cas-Geminin fusion protein and guide RNA to edit the target sequence.

Embodiment 44. A fusion protein, comprising: a) at least one Cas protein selected from the group consisting of wild-type Cas9, SaCas9, NmCas9, St1Cas9, HiFiCas9, wild-type Cas12, Cas12a, Cas12f, Cas13, Cas3, Caf1, AsCas12a, LbCas12a, and MAD7; and b) at least one Geminin variant comprising amino acids 2-60, 2-50, 2-40, 5-60, 5-50, 5-40, 10-60, 10-50, 10-40, 15-60, 15-50, 15-40, 18-60, 18-50, 18-40, and 18-36 of Geminin, wherein the amino acids are numbered from the first amino acid of the N-terminus of Geminin, wherein the at least one Geminin variant is fused to the C-terminus of the Cas protein, thereby forming the fusion protein. Embodiment 45. The fusion protein of embodiment 44, wherein the at least one Cas protein is wild-type Cas9 or HiFiCas9. Embodiment 46. The fusion protein of any one of embodiments 44 45, wherein the at least one Geminin variant is the Geminin variant comprising amino acids 2-60 of Geminin (Geminin 2-60). Embodiment 47. The fusion protein of any one of embodiments 44-46, wherein the at least one Geminin variant is the Geminin variant comprising amino acids 18-36 of Geminin (Geminin 18-36). Embodiment 48. The fusion protein of any one of embodiments 44-47, wherein the at least one Cas protein is wild-type Cas9 or HiFi Cas9. Embodiment 49. The fusion protein of any one of embodiments 44-48, wherein the at least one Geminin variant is the Geminin variant comprising amino acids 2-60 of Geminin (Geminin 2-60). Embodiment 50. The fusion protein of any one of embodiments 44-49, wherein the at least one Geminin variant is the Geminin variant comprising amino acids 18-36 of Geminin (Geminin 18-36). Embodiment 51. The fusion protein of any one of embodiments 44-50, wherein the at least one Cas protein is wild-type Cas9 with the amino acid sequence of SEQ ID NO: 28. Embodiment 52. The fusion protein of any one of embodiments 44-50 wherein the HiFi Cas9 has the amino acid sequence of SEQ ID NO: 26. Embodiment 53. The fusion protein of any one of embodiments 44-52, wherein the HiFi Cas9-Geminin 2-60 is encoded by the nucleic acid sequence of SEQ ID NO: 41. Embodiment 54. The fusion protein of any one of embodiments 44-53, wherein the HiFi Cas9-Geminin 2-40 is encoded by the nucleic acid sequence of SEQ ID NO: 42. Embodiment 55. The fusion protein of any one of embodiments 44-54 wherein the CRISPR-Cas is encoded by a nucleotide sequence of SEQ ID NO: 3. Embodiment 56. The fusion protein of any one of embodiments 44-55 wherein the CRISPR-Cas has the amino acid sequence of SEQ ID NO: 26. Embodiment 57. The fusion protein of any one of embodiments 44-56 wherein the Geminin 18-36 is encoded by a nucleotide sequence of SEQ ID NO: 6. Embodiment 58. The fusion protein of any one of embodiments 44-57 wherein the Geminin 18-36 has the amino acid sequence of SEQ ID NO: 36. Embodiment 59. The fusion protein of any one of embodiments 44-58 wherein the CRISPR-Cas-Geminin 18-36 is encoded by a nucleotide sequence of SEQ ID NO: 4. Embodiment 60. The fusion protein of any one of embodiments 44-59 wherein the CRISPR-Cas-Geminin 18-36 has the amino acid sequence of SEQ ID NO: 29.

Embodiment 61. An isolated mRNA encoding the fusion protein of any one of Embodiment embodiments 44-60.

Embodiment 62. A method for delivering a CRISPR system to a mammalian cell, comprising: (a) providing a CRISPR system comprising i. a CRISPR-Cas system guide RNA (“gRNA”) that hybridizes with a target sequence; and ii. an mRNA encoding the fusion protein of any one of embodiments 44-60; wherein components (i) and (ii) are located on same or different vectors of the system, (c) introducing the CRISPR system into the mammalian cell using electroporation, wherein the CRISPR system binds to and edits a target sequence within the genome of the mammalian cell.

### BRIEF DESCRIPTION OF SEVERAL VIEWS OF THE DRAWINGS

The invention will be described in conjunction with the following drawings in which like reference numerals designate like elements and wherein:

FIG. 1. (FIG. 1A) Schematic of proteins encoded by mRNA. The dashes (not drawn to scale) denote the amino acid sequence GGGGS (SEQ ID NO: 22). Gem 18-36 denotes Geminin amino acids 18-36, HA tag denotes hemagglutinin (HA) epitope tag, and NLS denotes nuclear localization sequence. K562 (FIG. 1B-FIG. 1C), HEK293 (FIG. 1D-FIG. 1E), and U2OS (FIG. 1F-FIG. 1G) cells were nucleofected with the indicated sgRNAs and HiFi Cas9 (dark gray bars) or HiFi Cas9-Geminin 18-36 (light gray bars) mRNA. Genomic DNA was isolated from all samples, NGS libraries were prepared using rhAmpSeq CRISPR library preparation protocol, and libraries were sequenced on an Illumina NextSeq 2000. Indels at on- and off-target sites were determined using CRISPy bioinformatic analysis (mean±SD, n=3).

FIG. 2. (FIG. 2A) Schematic of proteins encoded by mRNA. The dashes (not drawn to scale) denote the amino acid sequence GGGGS (SEQ ID NO: 22). Gem 18-36 denotes Geminin amino acids 18-36, HA tag denotes hemagglutinin (HA) epitope tag, and NLS denotes nuclear localization sequence. (FIG. 2B-2C) K562 cells were nucleofected with TRAC 3 sgRNA (FIG. 2B) or HPRT 38087 sgRNA (FIG. 2C), and HiFi Cas9 (dark gray lines) or HiFi Cas9-Geminin 18-36 (light gray lines) mRNA. (FIG. 2D-2E) U2OS cells were nucleofected with TRAC 3 sgRNA (FIG. 2D) or HPRT 38087 sgRNA (FIG. 2E), and HiFi Cas9 (dark gray lines) or HiFi Cas9-Geminin 18-36 (light gray lines) mRNA. Genomic DNA was isolated from all samples, NGS libraries were prepared using rhAmpSeq CRISPR library preparation protocol, and libraries were sequenced on an Illumina MiSeq System. Indels were determined using CRISPy bioinformatic analysis (mean±SD, n=3).

FIG. 3. (FIG. 3A) Schematic of proteins encoded by mRNA. The dashes (not drawn to scale) denote the amino acid sequence GGGGS (SEQ ID NO: 22). Gem 18-36 denotes Geminin amino acids 18-36, HA tag denotes hemagglutinin (HA) epitope tag, and NLS denotes nuclear localization sequence. (FIG. 3B) K562 cells were nucleofected with TRAC sgRNA and 1.4 μg of HiFi Cas9 (dark gray bar) or HiFi Cas9-Geminin 18-36 (light gray bar) mRNA. Percent viability was normalized to mock nucleofected cells (white bar) (mean±SD, n=2). (FIG. 3C) K562 cells were nucleofected with HPRT 38087 sgRNA and HiFi Cas9 (dark gray line) or HiFi Cas9-Geminin 18-36 (light gray line) mRNA. Percent viability was normalized to mock nucleofected cells (mean±SD, n=3).

FIG. 4. (FIG. 4A) Schematic of proteins encoded by mRNA. The dashes (not drawn to scale) denote the amino acid sequence GGGGS (SEQ ID NO: 22). Gem 18-36 denotes Geminin amino acids 18-36 and NLS denotes nuclear localization sequence. (FIG. 4B-FIG. 4D) K562 (FIG. 4B), HEK293 (FIG. 4C), or U20S (FIG. 4D) cells were nucleofected with 4.8 μM (4B) or 2.5 μM (4C-4D) HPRT 38087 sgRNA, and HiFi Cas9 (dark gray lines) or HiFi Cas9-Geminin 18-36 (light gray lines) mRNA. (FIG. 4E-FIG. 4F) HEK293 (FIG. 4E) or U20S (FIG. 4F) cells were nucleofected with 3.5 μM TRAC 3 sgRNA, and HiFi Cas9 (dark gray lines) or HiFi Cas9-Geminin 18-36 (light gray lines) mRNA. (FIG. 4G-FIG. 4H) HEK293 (FIG. 4G) or U20S (FIG. 4H) cells were nucleofected with 2.5 μM TRAC 8 sgRNA, and HiFi Cas9 (dark gray lines) or HiFi Cas9-Geminin 18-36 (light gray lines) mRNA. Genomic DNA was isolated from all samples, NGS libraries were prepared using rhAmpSeq CRISPR library preparation protocol, and libraries were sequenced on an Illumina MiSeq System. Indels were determined using CRISPy bioinformatic analysis (mean±SD, n=3).

FIG. 5. (FIG. 5A) Schematic of proteins encoded by mRNA. The dashes (not drawn to scale) denote the amino acid sequence GGGGS (SEQ ID NO: 22). Gem 18-36 denotes Geminin amino acids 18-36 and NLS denotes nuclear localization sequence. K562 (FIG. 5B-FIG. 5C), HEK293 (FIG. 5D-FIG. 5E), and U20S (FIG. 5F-FIG. 5G) cells were nucleofected with the indicated sgRNAs and Cas9 (dark gray bars) or Cas9-Geminin 18-36 (light gray bars) mRNA. 1 μg of mRNA was used for TRAC 3 nucleofections, while 0.625 μg of mRNA was used for all others. Genomic DNA was isolated from all samples, NGS libraries were prepared using rhAmpSeq CRISPR library preparation protocol, and libraries were sequenced on an Illumina NextSeq 2000. Indels at on- and off-target sites were determined using CRISPy bioinformatic analysis (mean±SD, n=3).

FIG. 6. (FIG. 6A) Schematic of proteins encoded by mRNA. The dashes (not drawn to scale) denote the amino acid sequence GGGGS (SEQ ID NO: 22). Gem 18-36 denotes Geminin amino acids 18-36 and NLS denotes nuclear localization sequence. (FIG. 6B-FIG. 6C) U20S cells were nucleofected with TRAC 3 (FIG. 6B) or AAVS1 (FIG. 6C) sgRNA, and Cas9 (dark gray lines) or Cas9-Geminin 18-36 (light gray lines) mRNA. The solid lines denote indels at the on-target site, and the dashed lines denote indels at an off-target site. Genomic DNA was isolated from all samples, NGS libraries were prepared using rhAmpSeq CRISPR library preparation protocol, and libraries were sequenced on an Illumina MiSeq System. Indels at on- and off-target sites were determined using CRISPy bioinformatic analysis (mean±SD, n=3).

FIG. 7. (FIG. 7A) Schematic of proteins encoded by mRNA. The dashes (not drawn to scale) denote the amino acid sequence GGGGS (SEQ ID NO: 22). Gem 18-36 denotes Geminin amino acids 18-36 and NLS denotes nuclear localization sequence. (FIG. 7B) U20S cells were nucleofected with 4.8 μM TRAC 3 sgRNA and Cas9 (dark gray line), Cas9-Geminin 18-36 (light gray line), Cas9-Geminin 18-36 L26A (red line), or Cas9-Geminin 18-36 K27R (blue line) mRNA. (FIG. 7C-FIG. 7E) U20S (FIG. 7C) or HEK293 (FIG. 7D-FIG. 7E) cells were nucleofected with 4.8 μM TRAC 3 sgRNA (FIG. 7C), 3.5 μM TRAC 3 sgRNA (FIG. 7D), or 2.5 μM HPRT 38087 sgRNA (FIG. 7E), and HiFi Cas9 (light gray line), HiFi Cas9-Geminin 18-36 (light gray line), or HiFi Cas9-Geminin 18-36 L26A (red line) mRNA. (FIG. 7F-FIG. 7G) induced pluripotent stem cells (iPSCs) were nucleofected with 2 μM AR (FIG. 7F) or AAVS1 (FIG. 7G) sgRNA, and HiFi Cas9 (gray bars/line) or HiFi Cas9-Geminin 18-36 L26A (red bars/line) mRNA. Genomic DNA was isolated from all samples, NGS libraries were prepared using rhAmpSeq CRISPR library preparation protocol, and libraries were sequenced on an Illumina NextSeq 2000. Indels were determined using CRISPy bioinformatic analysis (mean±SD, n=3).

FIG. 8A K562 cells were nucleofected with mRNA encoding the indicated HiFi Cas9-Geminin proteins and indels were assessed by next-generation sequencing (NGS). FIG. 8B denotes the proteins encoded by the mRNA that was introduced into K562 cells to generate indels.

### DETAILED DESCRIPTION

The Clustered Regularly Interspaced Short Palindromic Repeats and CRISPR-associated protein 9 (CRISPR-Cas9) system is a tool in genetic engineering to precisely edit genes within organisms. CRISPR-Cas systems are native to bacteria and Archaea to provide adaptive immunity against viruses and plasmids. CRISPR are sequences of DNA in bacterial genomes that store fragments of viral DNA. If the bacterium survives a viral attack, it incorporates a piece of the virus's genetic material into its own DNA. These stored sequences help the bacteria recognize and destroy a virus or plasmid. Cas9 is an enzyme that cuts DNA at specific sites. It's guided by a piece of RNA, known as a guide RNA (gRNA), which matches a specific DNA sequence that scientists want to modify. The gRNA is complementary to the target DNA sequence. This gRNA will guide the Cas9 protein to the exact location in the genome. Once guided to the target location, the Cas9 enzyme cuts the DNA at that precise spot. After Cas9 cuts the DNA, the cell attempts to repair the break. In mammalian cells repair processes can be error-prone, and inaccurate break repair leads to mutations that can disrupt the function of the targeted gene. However, if an exogenous repair template with the appropriate DNA sequence is also introduced into cells, the cells may incorporate this new sequence into the genome during the repair process.

There are three classes of CRISPR-Cas systems that could potentially be adapted for research and therapeutic reagents, but Type-II CRISPR systems have a desirable characteristic in utilizing a single CRISPR associated (Cas) nuclease (specifically Cas9) in a complex with the appropriate guide RNAs-either a 2-part RNA system similar to the natural complex in bacteria comprising a CRISPR-activating RNA:trans-activating crRNA (crRNA:tracrRNA) pair or an artificial chimeric single-guide-RNA (sgRNA)—to mediate double-stranded cleavage of target DNA. In mammalian systems, these RNAs have been introduced by transfection of DNA cassettes containing RNA Pol III promoters (such as U6 or H1) driving RNA transcription, viral vectors, and single-stranded RNA following in vitro transcription (see Xu, T., et al., Appl Environ Microbiol, 2014. 80(5): p. 1544-52).

The CRISPR-Cas9 system is utilized in genomic engineering as follows: a portion of the crRNA hybridizes to a target sequence, a portion of the tracrRNA hybridizes to a portion of the crRNA, and the Cas9 nuclease binds to the entire complex and directs cleavage. Cas9 contains two domains homologous to endonucleases HNH and RuvC, wherein the HNH domain cleaves the DNA strand complementary to the crRNA and the RuvC-like domain cleaves the noncomplementary strand. This results in a double-stranded break in genomic DNA. When repaired by non-homologous end joining (NHEJ) the break may be shifted by 1 or more bases, leading to disruption of the natural DNA sequence and in many cases leading to a frameshift mutation if the event occurs in the coding exon of a protein-coding gene. The break may also be repaired by homologous recombination (HR), which can permit insertion of new genetic material into the Cas9 cut site if an exogenous donor template is also introduced into cells.

The CRISPR/Cas9 components or CRISPR/Cpf1 components can be introduced into the cell using various approaches. Examples include plasmid or viral expression vectors (which lead to endogenous expression of either Cas9/Cpf1, the gRNAs (crRNA for Cpf1), or both), Cas9 or Cpf1 mRNA with separate gRNA/crRNA transfection, or delivery of the Cas9 or Cpf1 protein with the gRNA or crRNAs as a ribonucleoprotein (RNP) complex (see Kouranova et al., Hum Gen Ther (2016) 27(6):464-475). Each approach leads to different time-frames of availability of the active CRISPR system in the transfected cell.

There are two major DNA repair pathways for gene editing after DNA cleavage by Cas9: non-homologous end joining (NHEJ) and homology-directed repair (HDR). NHEJ can introduce random insertions or deletions (indels) in the target DNA sequence and is useful for gene knockout. By contrast, gene knock-in is dependent on HDR, and introduction of a donor template that has sequences homologous to sequences that flank the Cas9 cleavage site can result in donor template genomic integration.

Geminin prevents DNA re-replication by inhibiting a DNA replication licensing factor called Cdt1, and is expressed during S,G2, and M phases of the cell cycle. In G1 phase, Geminin is not expressed due to APC^{Cdh1}-dependent ubiquitination and degradation. Previous studies hypothesized that fusion of WT Cas9 to human Geminin amino acids 1-110 (Cas9-Gem) would result in G1-dependent Cas9-Gem degradation and increased knock-in percentages of exogenous donor templates; however, results of genome editing with Cas9-Gem showed only a modest, or no increase in exogenous donor knock-in rates.

The Cas-Geminin fusion variants as disclosed herein may comprise any of the available Cas proteins, or a range of Geminin amino acid sequences. CRISPR-associated (Cas) proteins useful in certain embodiments as disclosed herein may include, for example, Cas9, primarily from Streptococcus pyogenes (SpCas9); SaCas9 (Staphylococcus aureus); NmCas9 (Neisseria meningitidis); St1Cas9 (Streptococcus thermophilus); Cas9 Nickase Variants (Cas9n), which are Cas9 modified to create single-strand cuts (nicks) instead of double-strand breaks; Dead Cas9 (dCas9), A catalytically inactive form of Cas9 which is used for gene regulation and visualization, as it can bind to DNA without cutting it; Cas12 (Cpf1) derived from Francisella novicida (FnCpf1) and Acidaminococcus (AsCpf1); Cas12a recognizes a T-rich PAM, useful for AT-rich genomes; Cas12b, suitable for viral delivery systems; Cas12f (Cpf1 Mini); AsCas12a; LbCas12a; MAD7 which is a CRISPR-associated protein that belongs to the Cas12 family (a Type V CRISPR system); Cas13 which targets RNA instead of DNA; Cas13a; Cas13b; Cas13d; Cas3; Csf1, Type III (RNA-Targeting); and/or Csm and Cmr Complexes.

The Cas-Geminin fusions as disclosed herein may comprise HiFi Cas9, which refers to a high-fidelity version of Cas9, an enzyme that plays a key role in the CRISPR gene-editing system. Cas9 is used to create targeted double-strand breaks in DNA, allowing for precise gene editing. However, standard Cas9 can sometimes lead to off-target effects, where unintended parts of the genome are cut, leading to potential errors in gene editing. HiFi Cas9 is a modified version of Cas9 with reduced off-target activity that retains on-target gene-editing efficiency. This modification typically involves changing certain amino acids in the enzyme to improve its specificity. HiFi Cas9 thus offers more accurate gene editing, making it highly valuable in therapeutic applications, such as gene therapy and disease research, where minimizing unintended mutations is crucial.

HiFi Cas9 was developed as an alternative to Cas9 to create an enzyme that maintained potent on-target editing activity but had reduced off-target editing activity. However, in some instances, use of HiFi Cas9 also results in decreased on-target editing (11). Therefore, as HiFi Cas9-Geminin 18-36 generates higher indel percentages at on-target sites compared to HiFi Cas9 while generating similar indel levels at off-target sites, HiFi Cas9-Geminin 18-36 may be a superior alternative to HiFi Cas9 in experiments that require high editing fidelity. By contrast, Cas9-Geminin 18-36, when compared to Cas9, causes an increase in indels at both on- and off-target sites, which indicates that fusion of Geminin 18-36 to Cas9 is unlikely to be as useful for experiments where precise editing is required.

Cas9 from Staphylococcus aureus (SaCas9) or Streptococcus pyogenes (SpCas9) are the two most used Cas9 variants for genome editing applications. However, because the aforementioned bacterial species are common human pathogens, many people possess adaptive immunity towards both Sa- and SpCas9 (12). Pre-existing adaptive immunity is likely to limit the therapeutic potential of Cas9 in in vivo settings and may also be a source of toxicity. As such, use of HiFi Cas9-Geminin 18-36 instead of HiFi Cas9 may be useful for limiting patient immune responses as in some cases, compared to HiFi Cas9 mRNA, the HiFi Cas9-Geminin 18-36 mRNA dose can be halved and still generate equivalent or higher indel percentages.

CRISPR-Geminin is a specific variant of the broader CRISPR-Cas9 system, designed to improve the precision and efficiency of gene editing. It leverages the properties of Geminin, which is involved in regulating DNA replication during the cell cycle. By linking the CRISPR-Cas9 system with cell cycle control mechanisms, CRISPR-Geminin aims to enhance gene editing outcomes. Geminin is a naturally occurring protein that plays a critical role in the regulation of DNA replication. It prevents DNA replication from happening more than once per cell cycle by inhibiting a protein called Cdt1, which is involved in DNA replication licensing. Geminin is only expressed during specific cell cycle phases (primarily the S and G2 phases, when DNA is being replicated or has already been replicated). One of the main challenges in CRISPR-Cas9 editing is controlling how the cell repairs the DNA after Cas9 makes a double-strand break. Cells primarily use one of two pathways for double-strand break repair: non-homologous end joining (NHEJ): A fast but less accurate repair mechanism, which can lead to small insertions or deletions at the cut site. This can result in gene knockouts but is not ideal for precise gene editing; or homology-directed repair (HDR): A more precise, template-based repair mechanism that uses a DNA template to repair the break. This pathway allows introduction of specific changes to the DNA sequence, but it is less active and only occurs during certain phases of the cell cycle (mainly in the S and G2 phases when the DNA is actively being replicated or has already been replicated).

In certain embodiments as disclosed herein, (HiFi) Cas9-Geminin 18-36 can be used in genome editing for the generation of knock-out or knock-in cell lines. In addition, fusion of Geminin 18-36 to Cas9 nickases may improve the function of base and/or prime editors, and fusion of Geminin 18-36 to other genome editing enzymes such as MAD7 may increase desired editing outcomes too.

The Cas-Geminin fusion variants as disclosed herein may comprise full-length Geminin. The Cas-Geminin fusion variants as disclosed herein may comprise Geminin amino acid sequences. In exemplary embodiments as disclosed herein, the Geminin fusions may comprise a range of amino acids from Geminin. In exemplary embodiments as disclosed herein, the Geminin fusions may comprise a range of amino acids from Geminin such as amino acids 2-60, 2-50, 2-40, 5-60, 5-50, 5-40, 10-60, 10-50, 10-40, 15-60, 15-50, 15-40, 18-60, 18-50, 18-40, and 18-36 of Geminin (numbered from the first amino acid of the N-terminus of Geminin).

CRISPR guide RNA (gRNA) directs the Cas9 enzyme to a specific location in the genome where DNA cleavage may then occur. The gRNA is designed to match a target DNA sequence, ensuring the CRISPR-Cas9 system edits only the intended site. The guide RNA is made up of two main parts: CRISPR RNA (crRNA) which is a sequence of about 20 nucleotides that is complementary to the target DNA sequence. Its primary function is to guide the Cas9 protein to the exact location in the genome where the DNA cut should be made; and Trans-activating CRISPR RNA (tracrRNA) which aids in forming a stable complex with the Cas9 enzyme. It is necessary for the activation of the Cas9 protein, enabling it to perform its function as a molecular scissors.

A CRISPR eukaryotic expression cassette typically consists of several elements that together allow the CRISPR system to function efficiently within eukaryotic cells. These elements include the necessary components for gene editing, such as the Cas protein (usually Cas9) and the guide RNA (gRNA) system. In certain embodiments as disclosed herein, additional components for a eukaryotic CRISPR expression cassette may include a promoter for Cas protein expression, such as a CMV (Cytomegalovirus) promoter, EF1α (Elongation factor-la) promoter, Ubiquitin C (UbC) promoter, or Tissue-specific promoters for targeted Cas9 expression, e.g., neuron-specific promoters like Synapsin (Syn) or liver-specific like Albumin promoter. In certain embodiments as disclosed herein, additional components for a eukaryotic CRISPR expression cassette may include Cas protein coding sequence, such as HiFi Cas, SpCas9, Cpf1/Cas12a, SaCas9, or other Cas9 variants. In certain embodiments as disclosed herein, additional components for a eukaryotic CRISPR expression cassette may include nuclear localization signals (NLS) to ensure proper transport of the Cas9 protein into the nucleus of the eukaryotic cell. In certain embodiments as disclosed herein, additional components for a eukaryotic CRISPR expression cassette may include a promoter for gRNA Expression, such as a U6 promoter, an H1 promoter, or a Tissue-specific Pol II promoter. In certain embodiments as disclosed herein, additional components for a eukaryotic CRISPR expression cassette may include a guide RNA (gRNA) expression Unit, such as a gRNA scaffold or multiplexing gRNAs. In certain embodiments as disclosed herein, additional components for a eukaryotic CRISPR expression cassette may include a polyadenylation signal (pA), a selectable marker such as antibiotic resistance genes, or fluorescent markers. In certain embodiments as disclosed herein the expression cassette may include viral vector elements, such as lentiviral vectors, AAV (adeno-associated virus) vectors, or self-inactivating (SIN) elements. In certain embodiments as disclosed herein the expression cassette may include inducible systems, such as Tet-On/Tet-Off systems or CRISPRa/i systems, or insulator sequences such as cHS4 insulators.

A Cas NLS (nuclear localization signal) is a short peptide sequence that is added to CRISPR-associated (Cas) proteins, like Cas9, to help them enter the nucleus of a eukaryotic cell. Since gene-editing processes like CRISPR-Cas9 target DNA, which is located in the cell's nucleus, it is essential that Cas proteins efficiently reach this compartment. The nuclear localization signal (NLS) is a specific sequence of amino acids that is recognized by the cell's transport machinery. This sequence acts like a “tag” that signals the cell to transport the Cas protein into the nucleus. The NLS binds to nuclear import proteins, which then facilitate the passage of the Cas protein through nuclear pores, channels that regulate movement between the cytoplasm and the nucleus. By attaching an NLS to Cas proteins, scientists ensure these proteins reach the nucleus quickly and efficiently, enabling precise and effective gene editing of the target DNA.

There are several effective strategies for introducing the Cas-Geminin system as disclosed herein into target cells. Exemplary embodiments as disclosed herein include viral vectors, such as adeno-associated virus (AAV) which are widely used for CRISPR delivery because they are generally safe, induce minimal immune response, and have been approved in some gene therapy applications; lentivirus and retrovirus; and adenovirus.

Additional methods for introducing the Cas-Geminin system as disclosed herein into target cells includes, for example, lipid nanoparticles (LNPs) which are commonly used for delivering RNA-based therapies; electroporation, which involves applying an electrical field to create temporary pores in the cell membrane, allowing CRISPR components (like plasmids, ribonucleoprotein complexes, or mRNA) to enter the cell. Additional methods for introducing the Cas-Geminin system as disclosed herein into target cells includes, for example, ribonucleoprotein (RNP) complexes which involve directly delivering the Cas9 protein pre-complexed with guide RNA (gRNA) into cells, usually via electroporation or lipid-based transfection; lipid-based transfection agents (lipofection) uses lipid-based reagents to encapsulate CRISPR plasmids or RNP complexes and facilitate cellular uptake.

Other methods for introducing the Cas-Geminin system as disclosed herein into target cells includes, for example, physical methods such as microinjection to directly inject CRISPR components into cells, typically used in single-cell embryos or zygotes for generating transgenic animals; nanoneedles and microfluidics which can introduce CRISPR components with minimal damage to cells; and exosome-mediated delivery, which can be engineered to carry CRISPR/Cas components and target them to specific cells.

Next generation sequencing (NGS) allows rapid and high-throughput sequencing of DNA and RNA. Unlike earlier methods such as Sanger sequencing, which sequences one DNA fragment at a time, NGS enables the simultaneous sequencing of millions of DNA fragments, making it much faster, cheaper, and more efficient. In NGS, a DNA or RNA from the sample is extracted and fragmented into smaller pieces. These fragments are then attached to short synthetic DNA sequences called adapters, which are needed for binding to the sequencing platform. The DNA fragments with adapters are amplified (copied many times) to create a “library” of DNA fragments. This increases the amount of DNA available for sequencing. Most NGS platforms, like Illumina, use a method called “sequencing by synthesis.” Each fragment is attached to a solid surface and copied in place. Fluorescently-labeled nucleotides (A, T, C, and G) are added one by one. As they bind to the complementary strand, the machine detects the fluorescent signal, allowing the sequence of bases to be read. The massive amount of sequencing data is analyzed using bioinformatics tools. The overlapping DNA fragments are assembled back into their original sequence by aligning them to a reference genome or constructing new genomes (de novo sequencing). NGS is high throughput, since millions to billions of DNA fragments can be sequenced in parallel, producing vast amounts of data, is cost-effective, and can sequence entire genomes or large sets of genes in days, making it much faster than older sequencing methods.

The term “nucleic acid” refers to a nucleotide polymer, and unless otherwise limited, includes analogs of natural nucleotides that can function in a similar manner (e.g., hybridize) to naturally occurring nucleotides. The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acids, such as nucleic acid templates. Methods described herein additionally provide for the generation of isolated and at least partially purified nucleic acids and libraries of nucleic acids. Nucleic acids include but are not limited to those comprising DNA, RNA, circular RNA, cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like. More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses, for example, locked nucleic acids (LNAs) and unlocked nucleic acids (UNAs).

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “complementary” refers to the capacity for precise pairing between two nucleotides, i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules. The term “oligonucleotide,” as used herein, refer to polydeoxyribonucleotides (containing 2-deoxy-D-ribose), polyribonucleotides (containing D-ribose), and to any other type of polynucleotide which is an N glycoside of a purine or pyrimidine base (a single nucleotide is also referred to as a “base” or “residue”). There is no intended distinction in length between the terms “nucleic acid”, “oligonucleotide” and “polynucleotide”, and these terms can be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar, or phosphate backbone is modified as well as non-purine or non-pyrimidine nucleotide analogs. An oligonucleotide may comprise ribonucleotides, deoxyribonucleotides, modified nucleotides (e.g., nucleotides with 2′ modifications, synthetic base analogs, etc.) or combinations thereof.

The term “ribonucleotide” encompasses natural and synthetic, unmodified and modified ribonucleotides. Modifications include changes to the sugar moiety, to the base moiety, and/or to the linkages between ribonucleotides in the oligonucleotide.

The term “polypeptide” refers to any linear or branched peptide comprising more than one amino acid. Polypeptide includes protein or fragment thereof or fusion thereof, provided such protein, fragment or fusion retains a useful biochemical or biological activity. Fusion proteins typically include extra amino acid information that is not native to the protein to which the extra amino acid information is covalently attached. Such extra amino acid information may include tags that enable purification or identification of the fusion protein. Such extra amino acid information may include peptides that enable the fusion proteins to be transported into cells and/or transported to specific locations within cells. Examples of tags for these purposes include the following: AviTag, which is a peptide allowing biotinylation by the enzyme BirA so the protein can be isolated by streptavidin; Calmodulin-tag, which is a peptide bound by the protein calmodulin; polyglutamate tag, which is a peptide binding efficiently to anion-exchange resin such as Mono-Q; E-tag, which is a peptide recognized by an antibody; FLAG-tag, which is a peptide recognized by an antibody; HA-tag, which is a peptide from hemagglutinin recognized by an antibody, An HA sequence refers to a short amino acid sequence derived from the hemagglutinin (HA) protein of the influenza virus. It is commonly used as an epitope tag in molecular biology and biochemistry for detecting or purifying proteins. The HA sequence is typically recognized by specific antibodies, making it a useful tool for experiments requiring protein identification or manipulation. The HA tag is a small, well-defined peptide sequence, usually consisting of 9 amino acids: YPYDVPDYA (SEQ ID NO: 23); His-tag, which is typically 5-10 histidines bound by a nickel or cobalt chelate; Myc-tag, which is a peptide derived from c-myc recognized by an antibody; NE-tag, which is a novel 18-amino-acid synthetic peptide recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, which is a peptide derived from Ribonuclease A; SBP-tag, which is a peptide which binds to streptavidin; Softag 1, which is intended for mammalian expression; Softag 3, which is intended for prokaryotic expression; Strep-tag, which is a peptide which binds to streptavidin or the modified streptavidin called streptactin (Strep-tag II); TC tag, which is a tetracysteine tag that is recognized by FlAsH and ReAsH biarsenical compounds; V5 tag, which is a peptide recognized by an antibody; VSV-tag, a peptide recognized by an antibody; Xpress tag; Isopeptag, which is a peptide which binds covalently to pilin-C protein; SpyTag, which is a peptide which binds covalently to SpyCatcher protein; SnoopTag, a peptide which binds covalently to SnoopCatcher protein; BCCP (Biotin Carboxyl Carrier Protein), which is a protein domain biotinylated by BirA to enable recognition by streptavidin; Glutathione-S-transferase-tag, which is a protein that binds to immobilized glutathione; Green fluorescent protein-tag, which is a protein that is spontaneously fluorescent and can be bound by antibodies; HaloTag, which is a mutated bacterial haloalkane dehalogenase that covalently attaches to a reactive haloalkane substrate to allow attachment to a wide variety of substrates; Maltose binding protein-tag, a protein which binds to amylose agarose; Nus-tag; Thioredoxin-tag; and Fc-tag, derived from immunoglobulin Fc domain, which allows dimerization and solubilization and can be used for purification on Protein-A Sepharose. Nuclear localization signals (NLS), such as those obtained from SV40, allow for proteins to be transported to the nucleus immediately upon entering the cell. Given that the native Cas9 protein is bacterial in origin and therefore does not naturally comprise a NLS motif, addition of one or more NLS motifs to the recombinant Cas9 protein is expected to show improved genome editing activity in eukaryotic cells where the target genomic DNA substrate resides in the nucleus. One skilled in the art would appreciate these various fusion tag technologies, the particular amino acid sequences involved, as well as how to make and use fusion proteins that include them.

When a group of substituents is disclosed herein, it is understood that all individual members of those groups and all subgroups and classes that can be formed using the substituents are disclosed separately. When a Markush group or other grouping is used herein, all individual members of the group and all combinations and possible subcombinations of the group are intended to be individually included in the disclosure. As used herein, “and/or” means that one, all, or any combination of items in a list separated by “and/or” are included in the list; for example, “1, 2 and/or 3” is equivalent to “1, 2, 3, 1 and 2, 1 and 3, 2 and 3, or 1, 2, and 3”.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The disclosure as illustratively described herein suitably may be practiced in the absence of any element, elements, limitation, or limitations which is not specifically disclosed herein.

As used herein and in the appended claims, the singular forms “a”, “an”, and “the” include plural referents unless the context clearly dictates otherwise. Thus, for example, reference to “a method” includes a plurality of such methods and reference to “the nanoparticle” includes reference to one or more nanoparticles and equivalents thereof known to those skilled in the art, and so forth. The terms and expressions which have been employed are used as terms of description and not of limitation, and there is no intention in the use of such terms and expressions of excluding any equivalents of the features shown and described or portions thereof, but it is recognized that various modifications are possible within the scope as disclosed herein claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references throughout this application, for example patent documents, including issued or granted patents or equivalents and patent application publications, and non-patent literature documents or other source material are hereby incorporated by reference herein in their entireties, as though individually incorporated by reference. None is admitted to be prior art.

As used herein, the term “about” when used in conjunction with a stated numerical value or range has the meaning reasonably ascribed to it by a person skilled in the art, i.e., denoting somewhat more or somewhat less than the stated value or range.

The invention will be illustrated in more detail with reference to the following Examples, but it should be understood that the present invention is not deemed to be limited thereto.

### EXAMPLES

### Example 1

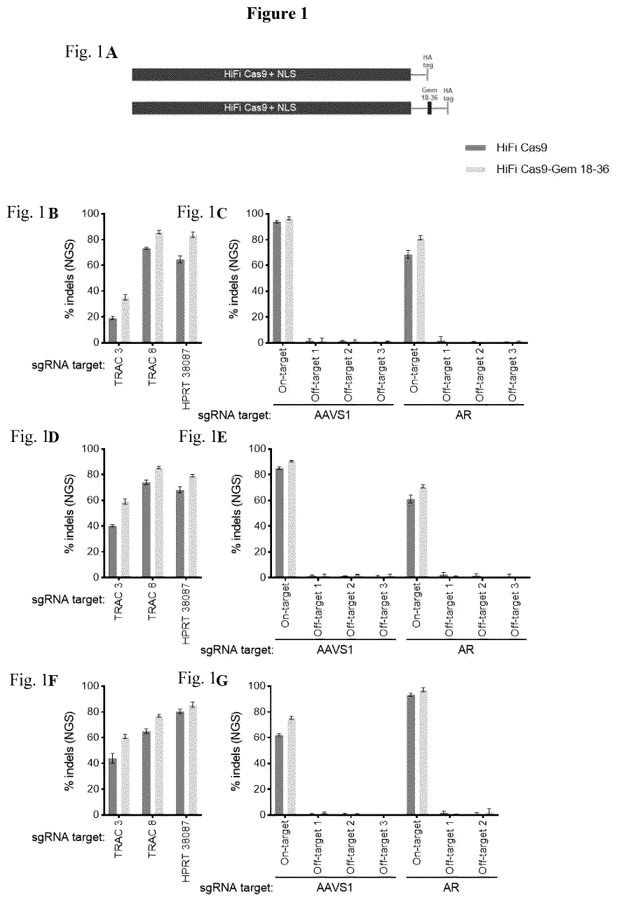

This Example demonstrates that fusion of Geminin amino acids 18-36 to HiFi Cas9 C-terminus increases on-target indel generation while minimally affecting off-target indel generation. See FIG. 1. K562 and HEK293 cells were harvested, resuspended in SF Cell Line Nucleofector Solution (Lonza #V4SC-2096), incubated with 4.8 μM sgRNA and 1 μg mRNA, and nucleofected with pulse code FF-120 (K562) or DS-150 (HEK293) using a 4D-Nucleofector 96-well Unit (Lonza #AAF-10035). U20S cells were harvested, resuspended in SE Cell Line Nucleofector Solution (Lonza #V4SC-1096), incubated with 4.8 μM sgRNA and 1 μg mRNA, and nucleofected with pulse code CM-104 using a 4D-Nucleofector 96-well Unit (Lonza #AAF-1003S). Cells were seeded into 96-well plates and cultured in DMEM+10% FBS (HEK293 and U20S) or IMDM+10% FBS (K562) for approximately 72 hours. Growth medium was then aspirated from cells, cells were washed with PBS, and genomic DNA was isolated from all samples using QuickExtract DNA Extraction Solution (BioSearch Technologies #SS000035-D2). Next-generation sequencing (NGS) libraries were generated for all samples following IDT's rhAmpSeq CRISPR library preparation protocol, and libraries were sequenced on a NextSeq 2000 (Illumina). Indels were quantitated using IDT's internal bioinformatic pipeline.

### Example 2

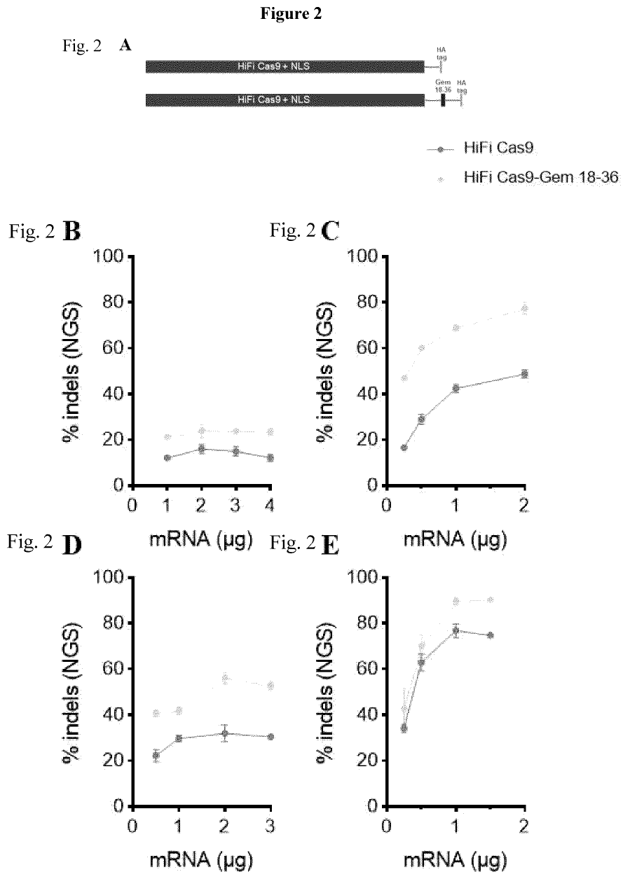

This Example demonstrates that HiFi Cas9-Geminin 18-36 generates higher percentages of indels than HiFi Cas9 in mRNA dose titration experiments. See FIG. 2. K562 and U20S cells were harvested, resuspended in SF (K562) Cell Line Nucleofector Solution (Lonza #V4SC-2096) or SE (U20S) Cell Line Nucleofector Solution (Lonza #V4SC-1096), incubated with 4.8 μM sgRNA and increasing mRNA amounts, and nucleofected with pulse code FF-120 (K562) or CM-104 (U20S) using a 4D-Nucleofector 96-well Unit (Lonza #AAF-1003 S). Cells were seeded into 96-well plates and cultured in DMEM+10% FBS (U20S) or IMDM+10% FBS (K562) for approximately 72 hours. Growth medium was then aspirated from cells, cells were washed with PBS, and genomic DNA was isolated from all samples using QuickExtract DNA Extraction Solution (BioSearch Technologies #SS000035-D2). Next-generation sequencing (NGS) libraries were generated for all samples following IDT's rhAmpSeq CRISPR library preparation protocol, and libraries were sequenced on a MiSeq System (Illumina). Indels were quantitated using IDT's internal bioinformatic pipeline.

### Example 3

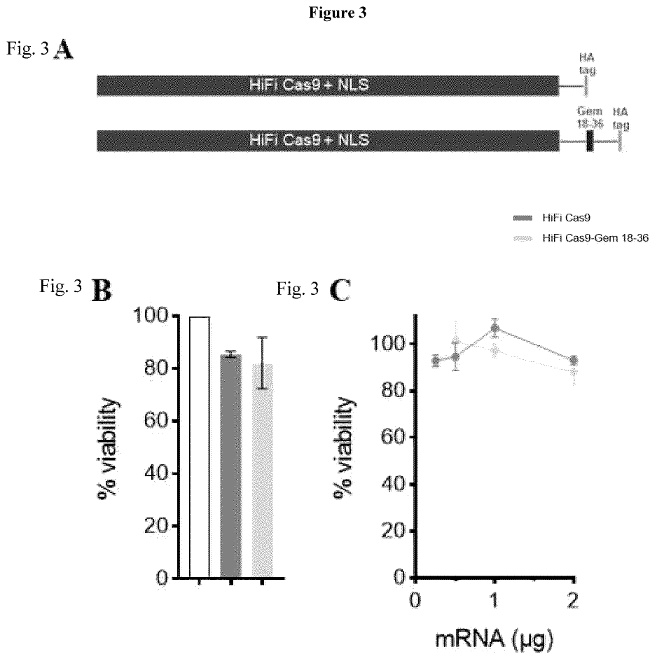

This Example demonstrates that nucleofection of cells with HiFi Cas9 or HiFi Cas9-Geminin 18-36 mRNA has similar effects on cell viability. See FIG. 3. K562 cells were harvested, resuspended in SF Cell Line Nucleofector Solution (Lonza #V4SC-2096), incubated with 4.8 μM sgRNA and a constant or increasing mRNA amount(s), and nucleofected with pulse code FF-120 using a 4D-Nucleofector 96-well Unit (Lonza #AAF-1003 S). Cells were seeded into 96-well plates and cultured in IMDM+10% FBS for approximately 72 hours. Presto Blue High Sensitivity (HS) Cell Viability Reagent (Invitrogen #P50201) was then added to the growth medium at a 1× concentration, cells were incubated with Presto Blue for 1 hour at 37° C., and cell viability was measured using a Tecan microplate reader.

### Example 4

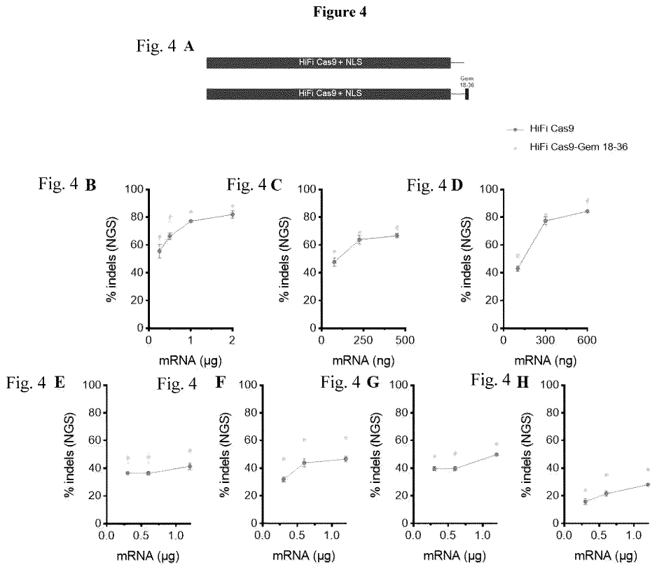

This Example demonstrates that HiFi Cas9-Geminin 18-36 generates higher percentages of indels than HiFi Cas9 in mRNA dose titration experiments when expressing proteins without epitope tags. See FIG. 4. K562 and HEK293 cells were harvested, resuspended in SF Cell Line Nucleofector Solution (Lonza #V4SC-2096), incubated with sgRNA (doses indicated in Brief Description of Several Views of the Drawings, above) and increasing mRNA doses, and nucleofected with pulse code FF-120 (K562) or DS-150 (HEK293) using a 4D-Nucleofector 96-well Unit (Lonza #AAF-10035). U20S cells were harvested, resuspended in SE Cell Line Nucleofector Solution (Lonza #V4SC-1096), incubated with sgRNA (doses indicated in Brief Description of Several Views of the Drawings) and increasing mRNA amounts, and nucleofected with pulse code CM-104 using a 4D-Nucleofector 96-well Unit (Lonza #AAF-1003 S). Cells were seeded into 96-well plates and cultured in DMEM+10% FBS (HEK293 and U20S) or IMDM+10% FBS (K562) for approximately 72 hours. Growth medium was then aspirated from cells, cells were washed with PBS, and genomic DNA was isolated from all samples using QuickExtract DNA Extraction Solution (BioSearch Technologies #SS000035-D2). Next-generation sequencing (NGS) libraries were generated for all samples following IDT's rhAmpSeq CRISPR library preparation protocol, and libraries were sequenced on an Illumina MiSeq System. Indels were quantitated using IDT's internal bioinformatic pipeline.

### Example 5

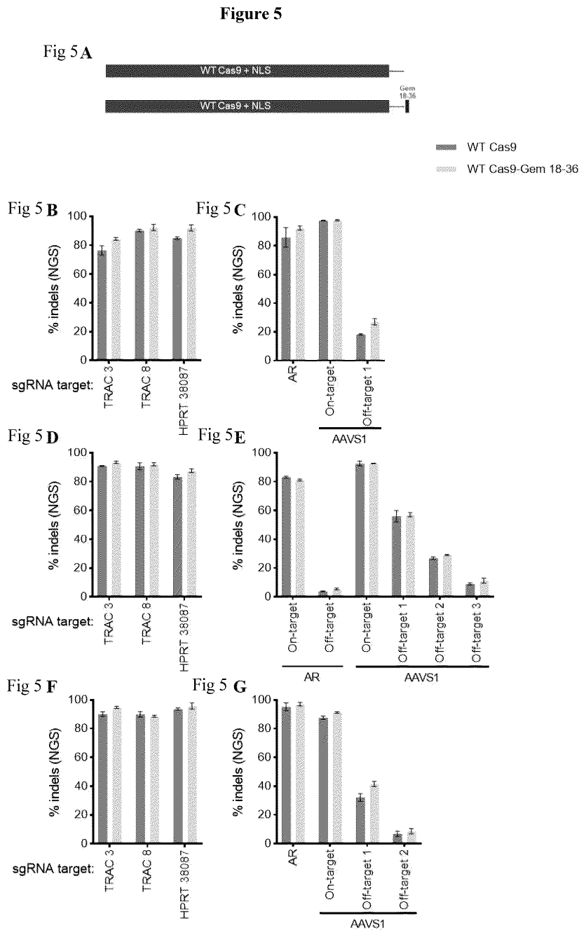

This Example demonstrates that fusion of Geminin amino acids 18-36 to Cas9 C-terminus increases on- and off-target indel generation. See FIG. 5. K562 and HEK293 cells were harvested, resuspended in SF Cell Line Nucleofector Solution (Lonza #V4SC-2096), incubated with 4.8 μM sgRNA and mRNA (doses indicated in Brief Description of Several Views of the Drawings), and nucleofected with pulse code FF-120 (K562) or DS-150 (HEK293) using a 4D-Nucleofector 96-well Unit (Lonza #AAF-1003S). U20S cells were harvested, resuspended in SE Cell Line Nucleofector Solution (Lonza #V4SC-1096), incubated with 4.8 μM sgRNA and mRNA (doses indicated in Brief Description of Several Views of the Drawings), and nucleofected with pulse code CM-104 using a 4D-Nucleofector 96-well Unit (Lonza #AAF-1003 S). Cells were seeded into 96-well plates and cultured in DMEM+10% FBS (HEK293 and U20S) or IMDM+10% FBS (K562) for approximately 72 hours. Growth medium was then aspirated from cells, cells were washed with PBS, and genomic DNA was isolated from all samples using QuickExtract DNA Extraction Solution (BioSearch Technologies #SS000035-D2). Next-generation sequencing (NGS) libraries were generated for all samples following IDT's rhAmpSeq CRISPR library preparation protocol, and libraries were sequenced on a NextSeq 2000 (Illumina). Indels were quantitated using IDT's internal bioinformatic pipeline.

### Example 6

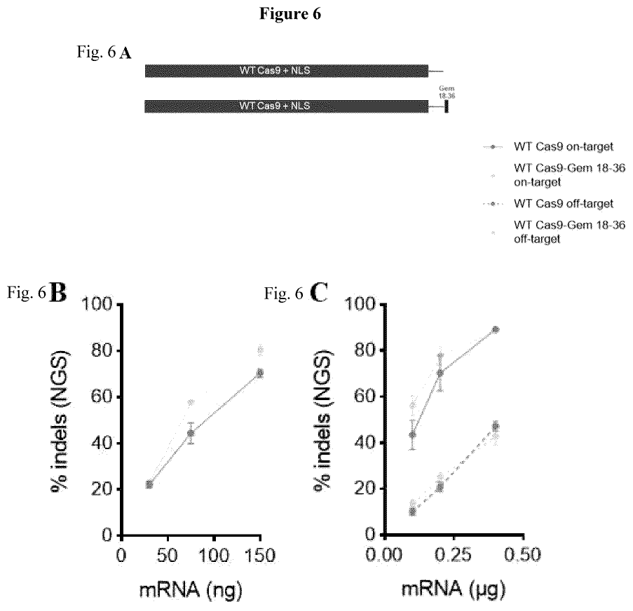

This Example demonstrates that Cas9-Geminin 18-36 generates higher percentages of indels than Cas9 in mRNA dose titration experiments. See FIG. 6. U20S cells were harvested, resuspended in SE Cell Line Nucleofector Solution (Lonza #V4SC-1096), incubated with 4.8 μM sgRNA and increasing mRNA doses, and nucleofected with pulse code CM-104 using a 4D-Nucleofector 96-well Unit (Lonza #AAF-10035). Cells were seeded into 96-well plates and cultured in DMEM+10% FBS for approximately 72 hours. Growth medium was then aspirated from cells, cells were washed with PBS, and genomic DNA was isolated from all samples using QuickExtract DNA Extraction Solution (BioSearch Technologies #SS000035-D2). Next-generation sequencing (NGS) libraries were generated for all samples following IDT's rhAmpSeq CRISPR library preparation protocol, and libraries were sequenced on an Illumina MiSeq System. Indels were quantitated using IDT's internal bioinformatic pipeline.

### Example 7

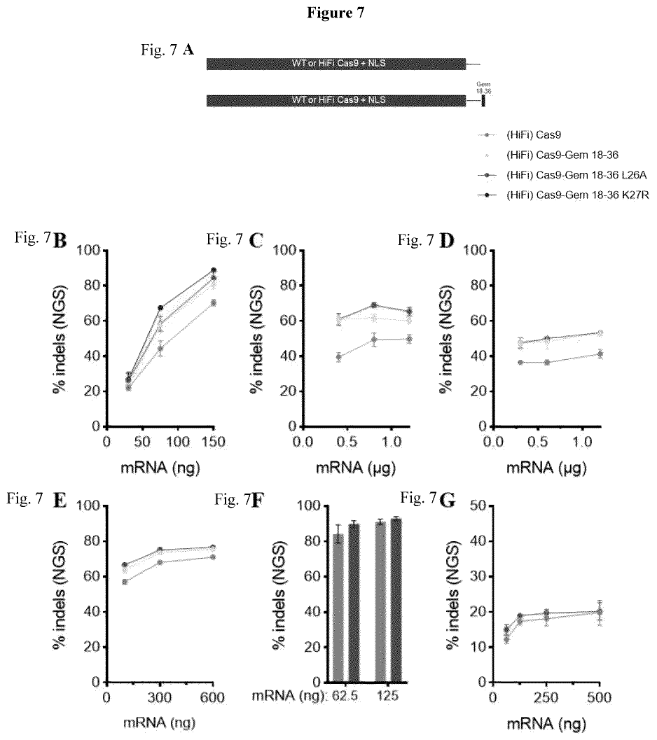

This Example demonstrates that fusion of Geminin amino acids 18-36 containing L26A or K27R mutation to (HiFi) Cas9 C-terminus sometimes further increases indel generation compared to (HiFi) Cas9-Geminin 18-36. See FIG. 7. HEK293 cells were harvested, resuspended in SF Cell Line Nucleofector Solution (Lonza #V4SC-2096), incubated with the indicated sgRNA and mRNA doses (denoted in Brief Description of Several Views of the Drawings), and nucleofected with pulse code DS-150 using a 4D-Nucleofector 96-well Unit (Lonza #AAF-10035). U20S cells were harvested, resuspended in SE Cell Line Nucleofector Solution (Lonza #V4SC-1096), incubated with the indicated sgRNA and mRNA doses (denoted in Brief Description of Several Views of the Drawings), and nucleofected with pulse code CM-104 using a 4D-Nucleofector 96-well Unit (Lonza #AAF-1003 S). Cells were seeded into 96-well plates and cultured in DMEM+10% FBS for approximately 72 hours. Induced pluripotent stem cells (iPSCs) were harvested, resuspended in P3 buffer (Lonza #V4XP-3032), incubated with 2 μM sgRNA and the indicated mRNA doses, and nucleofected with pulse code CA-137 using a 4D-Nucleofector 96-well Unit (Lonza #AAF-1003 S). iPSCs were seeded into 96-well plates and cultured in mTeSR medium with 1× CloneR 2 supplement (StemCell Technologies #100-0691) for 24 hours and then in mTeSR medium without supplement for an additional 4-5 days.

### Example 8

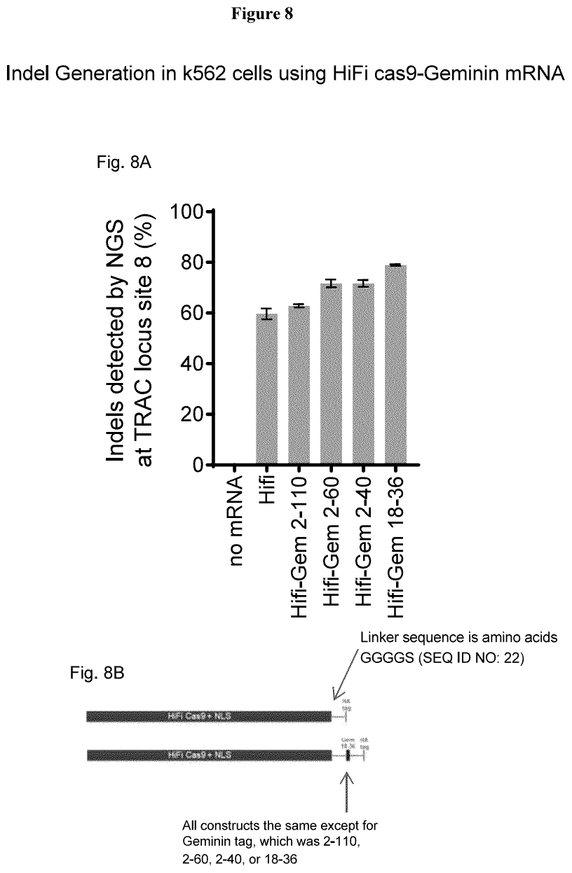

This Example demonstrates that indel generation in K562 cells by HiFi Cas9 can be increased by fusing various Geminin fragments to the HiFi Cas9 C-terminus. See FIG. 8. K562 cells were harvested, resuspended in SF Cell Line Nucleofector Solution (Lonza #V4SC-2096), incubated with 4.8 μM sgRNA and 1.4 μg mRNA, and nucleofected with pulse code FF-120 using a 4D-Nucleofector 96-well Unit (Lonza #AAF-1003S). Cells were seeded into 96-well plates and cultured in IMDM+10% FBS for approximately 72 hours. Growth medium was then aspirated from cells, cells were washed with PBS, and genomic DNA was isolated from all samples using QuickExtract DNA Extraction Solution (BioSearch Technologies #SS000035-D2). Next-generation sequencing (NGS) libraries were generated for all samples following IDT's rhAmpSeq CRISPR library preparation protocol, and libraries were sequenced on a NextSeq 2000 (Illumina). Indels were quantitated using IDT's internal bioinformatic pipeline.

While the invention has been described in detail and with reference to specific examples thereof, it will be apparent to one skilled in the art that various changes and modifications can be made therein without departing from the spirit and scope thereof.

### BACKGROUND REFERENCES

1. Maurissen T. L., Woltjen K. Synergistic gene editing in human iPS cells via cell cycle and DNA repair modulation. Nat Commun. 2020; 11(1):2876.
2. Wienert B., Nguyen D. N., Guenther A., Feng S. J., Locke M. N., Wyman S. K., Shin J., Kazane K. R., Gregory G. L., Carter M. A. M., Wright F., Conklin B. R., Marson A., Richardson C. D., Corn J. E. Timed inhibition of CDC7 increases CRISPR-Cas9 mediated templated repair. Nat Commun. 2020; 11(1):2109.
3. Lin S., Staahl B. T., Alla R. K., Doudna J. A. Enhanced homology-directed human genome engineering by controlled timing of CRISPR/Cas9 delivery. Elife. 2014; 3:e04766.
4. Canny M. D., Moatt N., Wan L. C. K., Fradet-Turcotte A., Krasner D., Mateos-Gomez P. A., Zimmermann M., Orthwein A., Juang Y., Zhang W., Noordermeer S. M., Seclen E., Wilsom M. D., Vorobyov A., Munro M., Ernst A., Ng T. F., Cho T., Cannon P. M., Sidhu S. S., Sicheri F., Durocher D. Inhibition of 53BP1 favors homology-dependent DNA repair and increases CRISPR-Cas9 genome-editing efficiency. Nat Biotechnol. 2018; 36(1):95-102.
5. McGarry T. J., Kirschner M. W. Geminin, an inhibitor of DNA replication, is degraded during mitosis. Cell. 1998; 93(6):1043-53.
6. Sakaue-Sawano A., Kurokawa H., Morimura T., Hanyu A., Hama H., Osawa H., Kashiwagi S., Fukami K., Miyata T., Miyoshi H., Imamura T., Ogawa M., Masai H., Miyawaki A. Visualizing spatiotemporal dynamics of multicellular cell-cycle progression. Cell. 2008; 132(3):487-98.
7. Gutschner T., Haemmerle M., Genovese G., Draetta G. F., Chin L. Post-translational Regulation of Cas9 during G1 Enhances Homology-Directed Repair. Cell Rep. 2016; 14(6):1555-1566.
8. Schimmel J., Munoz-Subirana N., Kool H., van Schendel R., van der Vlies S., Kamp J. A., de Vrij F. M. S., Kushner S. A., Smith G. C. M., Boulton S. J., Tijsterman M. Modulating mutational outcomes and improving precise gene editing at CRISPR-Cas9-induced breaks by chemical inhibition of end-joining pathways. Cell Rep. 2023; 42(2):112019.
9. Charpentier M., Khedler A. H. Y., Menoret S., Brion A., Lamribet K., Dardillac E., Boix C., Perrouault L., Tesson L., Geny S., De Cian A., Itier J. M., Anegon I., Lopez B., Giovannangeli C., Concordet J. P. CtIP fusion to Cas9 enhances transgene integration by homology-dependent repair. Nat Commun. 2018; 9(1):1133.
10. Howden S. E., McColl B., Glaser A., Vadolas J., Petrou S., Little M. H., Elefanty A. G., Stanley E. G. A Cas9 Variant for Efficient Generation of Indel-Free Knockin or Gene-Corrected Human Pluripotent Stem Cells. Stem Cell Rep. 2016; 7(3):508-517.

What is claimed is:

1. An engineered, non-naturally occurring Clustered Regularly Interspaced Short Palindromic Repeats (CRISPR)-Cas-Geminin system comprising:
a) a first regulatory element operable in a eukaryotic cell operably linked to at least one nucleotide sequence encoding a CRISPR-Cas system guide RNA (“gRNA”) that hybridizes with a target sequence;
b) a second regulatory element operable in a eukaryotic cell operably linked to a nucleotide sequence encoding a fusion protein comprising Geminin sequence amino acids fused to the C-terminus of a Cas protein, thereby forming a Cas-Geminin fusion protein;
c) wherein components (a) and (b) are located on same or different vectors of the system,
wherein the gRNA targets and hybridizes with the target sequence and the Cas-Geminin fusion protein cleaves the DNA molecule, wherein the Cas-Geminin fusion protein and the gRNA do not naturally occur together.

2. The system of claim 1, wherein the Cas-Geminin fusion protein comprises a Cas protein selected from the group consisting of wild-type Cas9, SaCas9, NmCas9, St1Cas9, HiFi-Cas9, wild-type Cas12, Cas12a, Cas12f, Cas13, Cas3, Caf1, AsCas12a, LbCas12a, and MAD7.

3. The system of any one of claims 1-2, wherein the Cas-Geminin fusion protein comprises at least one Geminin variant comprising amino acids 2-60, 2-50, 2-40, 5-60, 5-50, 5-40, 10-60, 10-50, 10-40, 15-60, 15-50, 15-40, 18-60, 18-50, 18-40, and 18-36 of Geminin, wherein the amino acids are numbered from the first amino acid of the N-terminus of Geminin that is fused to the C-terminus of the Cas protein, thereby forming the Cas-Geminin fusion protein.

4. The system of any one of claims 1-3 wherein the active CRISPR-Cas-Geminin fusion protein displays reduced off-target editing activity and maintains on-target editing activity relative to a wild-type CRISPR/Cas endonuclease system having the wild-type Cas9 protein of SEQ ID NO: 5.

5. The system of any one of claims 1-4 wherein the target sequence is in a DNA molecule in a eukaryotic cell, wherein the DNA molecule encodes, and the eukaryotic cell expresses, at least one gene product.

6. The system of any one of claims 1-5 wherein the CRISPR-Cas-Geminin fusion protein further comprises one or more Nuclear Localization Signals (NLS).

7. The system of any one of claims 1-6 wherein the CRISPR-Cas-Geminin fusion protein further comprises a tag selected from the group consisting of an HA tag, an AviTag, a Calmodulin-tag, a polyglutamate tag, an E-tag, and a FLAG-tag.

8. The system of any one of claims 1-7 wherein the CRISPR-Cas-Geminin fusion protein further comprises a linker sequence, optionally wherein the linker sequence has the amino acid sequence GGGGS (SEQ ID NO: 22).

9. The system of any one of claims 1-8 wherein the system alters expression of at least one gene product.

10. The system of any one of claims 1-9 wherein the Cas-Geminin fusion protein is codon optimized for expression in the eukaryotic cell.

11. The system of any one of claims 1-10 wherein the eukaryotic cell is a human cell.

12. The system of any one of claims 1-11 wherein the system increases expression of one or more gene products.

13. The system of any one of claims 1-12 wherein the system decreases expression of one or more gene products.

14. The system of any one of claims 1-13 wherein the CRISPR-Cas is encoded by a nucleotide sequence of SEQ ID NO: 3.

15. The system of any one of claims 1-14 wherein the CRISPR-Cas has the amino acid sequence of SEQ ID NO: 26.

16. The system of any one of claims 1-15 wherein the Geminin is Geminin 18-36 which is encoded by a nucleotide sequence of SEQ ID NO: 6.

17. The system of any one of claims 1-16 wherein the Geminin is Geminin 18-36 which has the amino acid sequence of SEQ ID NO: 36.

18. The system of any one of claims 1-17 wherein the Cas-Geminin fusion protein is Cas-Geminin 18-36 which is encoded by a nucleotide sequence of SEQ ID NO: 4.

19. The system of any one of claims 1-18 wherein the Cas-Geminin fusion protein is Cas-Geminin 18-36 which has the amino acid sequence of SEQ ID NO: 29.

20. A method of performing gene editing having reduced off-target editing activity and/or increased on-target editing activity, comprising:
contacting a candidate editing target site locus with an active CRISPR-Cas-Geminin system comprising:
a) a first regulatory element operable in a eukaryotic cell operably linked to at least one nucleotide sequence encoding a CRISPR-Cas system guide RNA (“gRNA”) that hybridizes with a target sequence;
b) a second regulatory element operable in a eukaryotic cell operably linked to a nucleotide sequence encoding a fusion protein comprising Geminin sequence amino acids fused to the C-terminus of Cas protein, thereby forming a Cas-Geminin fusion protein;
c) wherein components (a) and (b) are located on same or different vectors of the system,
wherein the gRNA targets and hybridizes with the target sequence and the Cas-Geminin fusion protein cleaves the DNA molecule wherein the Cas-Geminin fusion protein and the gRNA do not naturally occur together.

21. The method of claim 20, wherein the Cas-Geminin fusion protein comprises a Cas protein selected from the group consisting of wild-type Cas9, SaCas9, NmCas9, St1Cas9, HiFi-Cas9, wild-type Cas12, Cas12a, Cas12f, Cas13, Cas3, Caf1, AsCas12a, LbCas12a, and MAD7.

22. The method of any one of claims 20-21 wherein the Cas-Geminin fusion protein comprises at least one Geminin variant comprising amino acids 2-60, 2-50, 2-40, 5-60, 5-50, 5-40, 10-60, 10-50, 10-40, 15-60, 15-50, 15-40, 18-60, 18-50, 18-40, and 18-36 of Geminin, wherein the amino acids are numbered from the first amino acid of the N-terminus of Geminin that is fused to the C-terminus of the Cas protein, thereby forming a Cas-Geminin fusion protein.

23. The method of any one of claims 20-22 wherein the active CRISPR-Cas-Geminin fusion protein displays reduced off-target editing activity and maintained on-target editing activity relative to a wild-type CRISPR/Cas endonuclease system having the wild-type Cas9 protein of SEQ ID NO: 1.

24. The method of any one of claims 20-23 wherein the target sequence is in a DNA molecule in a eukaryotic cell, wherein the DNA molecule encodes, and the eukaryotic cell expresses, at least one gene product

25. The method of any one of claims 20-24 wherein the CRISPR-Cas-Geminin fusion protein further comprises one or more Nuclear Localization Signals (NLS).

26. The method of any one of claims 20-25 wherein the CRISPR-Cas-Geminin fusion protein further comprises a tag selected from the group consisting of an HA tag, an AviTag, a Calmodulin-tag, a polyglutamate tag, an E-tag, and a FLAG-tag.

27. The method of any one of claims 20-26 wherein the CRISPR-Cas-Geminin fusion protein further comprises a linker sequence, optionally wherein the linker sequence has the amino acid sequence GGGGS (SEQ ID NO: 22).

28. The method of any one of claims 20-27 wherein expression of at least one gene product is altered.

29. The method of any one of claims 20-28 wherein the Cas-Geminin fusion protein is codon optimized for expression in the eukaryotic cell.

30. The method of any one of claims 20-29 wherein the eukaryotic cell is a human cell.

31. The method of any one of claims 20-30 wherein the expression of one or more gene products is increased.

32. The method of any one of claims 20-31 wherein the expression of one or more gene products is decreased.

33. The method of any one of claims 20-32 wherein the CRISPR-Cas is encoded by a nucleotide sequence of SEQ ID NO: 3.

34. The method of any one of claims 20-33 wherein the CRISPR-Cas has the amino acid sequence of SEQ ID NO: 26.

35. The method of any one of claims 20-34 wherein the Geminin 18-36 is encoded by a nucleotide sequence of SEQ ID NO: 6.

36. The method of any one of claims 20-35 wherein the Geminin 18-36 has the amino acid sequence of SEQ ID NO: 36.

37. The method of any one of claims 20-36 wherein the CRISPR-Cas-Geminin 18-36 is encoded by a nucleotide sequence of SEQ ID NO: 4.

38. The method of any one of claims 20-37 wherein the CRISPR-Cas-Geminin 18-36 has the amino acid sequence of SEQ ID NO: 29.

39. A method of delivering the CRISPR system of any one of claims 1-19 to a mammalian cell, the method comprising the steps of:
(a) providing a first viral vector component encoding a CRISPR-Cas system guide RNA (“gRNA”) that hybridizes with a target sequence;
(b) providing a second viral vector component encoding a fusion protein comprising Geminin sequence amino acids fused to the C-terminus of Cas protein, thereby forming a Cas-Geminin fusion protein;
wherein components (a) and (b) are located on same or different vectors of the system; and
(c) transducing the mammalian cell with the viral vector(s) under conditions sufficient to express the Cas-Geminin fusion protein and the gRNA,
wherein the Cas-Geminin fusion protein and the gRNA form a complex that binds to and edits the target sequence.

40. A method for delivering the CRISPR system of any one of claims 1-19 to a mammalian cell, comprising:
(a) providing lipid nanoparticles encapsulating a CRISPR-Cas system guide RNA (“gRNA”) that hybridizes with a target sequence;
(b) providing lipid nanoparticles encapsulating an mRNA encoding a fusion protein comprising Geminin sequence amino acids fused to the C-terminus of Cas protein, thereby forming a Cas-Geminin fusion protein;
wherein components (a) and (b) are located on same or different vectors of the system,
(c) introduction of the mammalian cell with lipid nanoparticles under conditions sufficient to express the Cas-Geminin fusion protein and the gRNA,
wherein the Cas-Geminin fusion protein and guide RNA are expressed and form a complex to edit a specific target sequence in the cell's genome.

41. A method of performing gene editing having reduced off-target editing activity and/or increased on-target editing activity, comprising:
(a) providing a ribonucleoprotein (RNP) complex comprising the CRISPR system of any one of claims 1-19; and
(b) introducing the RNP complex into the mammalian cell using electroporation, wherein the CRISPR system binds to and edits a target sequence within the genome of the mammalian cell.

42. A method of delivering the CRISPR system of any one of claims 1-19 to a mammalian cell, comprising:
(a) preparing a lipofection reagent comprising a first regulatory element operable in a eukaryotic cell operably linked to at least one nucleotide sequence encoding a CRISPR-Cas system guide RNA (“gRNA”) that hybridizes with a target sequence;
(b) a second regulatory element operable in a eukaryotic cell operably linked to a nucleotide sequence encoding a fusion protein comprising Geminin sequence amino acids fused to the C-terminus of Cas protein, thereby forming a Cas-Geminin fusion protein;
wherein components (a) and (b) are located on the same or different vectors of the system, and
(c) applying the lipofection reagent to the mammalian cell,
wherein the Cas-Geminin fusion protein and guide RNA are expressed and form a complex to edit a specific target sequence in the cell's genome.

43. A method of targeted delivery of the CRISPR system of any one of claims 1-19 to a mammalian cell, comprising:
(a) preparing exosomes encapsulating a first nucleotide sequence encoding a CRISPR-Cas system guide RNA (“gRNA”) that hybridizes with a target sequence; and
(b) preparing exosomes encapsulating a second nucleotide sequence encoding a fusion protein comprising Geminin sequence amino acids fused to the C-terminus of Cas protein, thereby forming a Cas-Geminin fusion protein;
wherein components (a) and (b) are located on same or different vectors of the system; and
(c) delivering the engineered exosomes to the mammalian cell under conditions that allow the Cas-Geminin fusion protein and guide RNA to edit the target sequence.

44. A fusion protein, comprising:
a) at least one Cas protein selected from the group consisting of wild-type Cas9, SaCas9, NmCas9, St1Cas9, HiFiCas9, wild-type Cas12, Cas12a, Cas12f, Cas13, Cas3, Caf1, AsCas12a, LbCas12a, and MAD7; and
b) at least one Geminin variant comprising amino acids 2-60, 2-50, 2-40, 5-60, 5-50, 5-40, 10-60, 10-50, 10-40, 15-60, 15-50, 15-40, 18-60, 18-50, 18-40, and 18-36 of Geminin, wherein the amino acids are numbered from the first amino acid of the N-terminus of Geminin,
wherein the at least one Geminin variant is fused to the C-terminus of the Cas protein, thereby forming the fusion protein.

45. The fusion protein of claim 44, wherein the at least one Cas protein is wild-type Cas9 or HiFiCas9.

46. The fusion protein of any one of claims 44-45, wherein the at least one Geminin variant is the Geminin variant comprising amino acids 2-60 of Geminin (Geminin 2-60).

47. The fusion protein of any one of claims 44-46, wherein the at least one Geminin variant is the Geminin variant comprising amino acids 18-36 of Geminin (Geminin 18-36).

48. The fusion protein of any one of claims 44-47, wherein the at least one Cas protein is wild-type Cas9 or HiFi Cas9.

49. The fusion protein of any one of claims 44-48, wherein the at least one Geminin variant is the Geminin variant comprising amino acids 2-60 of Geminin (Geminin 2-60).

50. The fusion protein of any one of claims 44-49, wherein the at least one Geminin variant is the Geminin variant comprising amino acids 18-36 of Geminin (Geminin 18-36).

51. The fusion protein of any one of claims 44-50, wherein the at least one Cas protein is wild-type Cas9 with the amino acid sequence of SEQ ID NO: 28.

52. The fusion protein of any one of claims 44-50 wherein the HiFi Cas9 has the amino acid sequence of SEQ ID NO: 26.

53. The fusion protein of any one of claims 44-52, wherein the HiFi Cas9-Geminin 2-60 is encoded by the nucleic acid sequence of SEQ ID NO: 41.

54. The fusion protein of any one of claims 44-53, wherein the HiFi Cas9-Geminin 2-40 is encoded by the nucleic acid sequence of SEQ ID NO: 42.

55. The fusion protein of any one of claims 44-54 wherein the CRISPR-Cas is encoded by a nucleotide sequence of SEQ ID NO: 3.

56. The fusion protein of any one of claims 44-55 wherein the CRISPR-Cas has the amino acid sequence of SEQ ID NO: 26.

57. The fusion protein of any one of claims 44-56 wherein the Geminin 18-36 is encoded by a nucleotide sequence of SEQ ID NO: 6.

58. The fusion protein of any one of claims 44-57 wherein the Geminin 18-36 has the amino acid sequence of SEQ ID NO: 36.

59. The fusion protein of any one of claims 44-58 wherein the CRISPR-Cas-Geminin 18-36 is encoded by a nucleotide sequence of SEQ ID NO: 4.

60. The fusion protein of any one of claims 44-59 wherein the CRISPR-Cas-Geminin 18-36 has the amino acid sequence of SEQ ID NO: 29.

61. An isolated mRNA encoding the fusion protein of any one of claims 44-60.

62. A method for delivering a CRISPR system to a mammalian cell, comprising:
(a) providing a CRISPR system comprising
i. a CRISPR-Cas system guide RNA (“gRNA”) that hybridizes with a target sequence; and
ii. an mRNA encoding the fusion protein of any one of claims 44-60;
wherein components (i) and (ii) are located on same or different vectors of the system,
(c) introducing the CRISPR system into the mammalian cell using electroporation, wherein the CRISPR system binds to and edits a target sequence within the genome of the mammalian cell.


SEQUENCE LISTING (42 sequences)

SEQ ID NO: 1 - DNA, 7210 bp, synthetic construct
  source 1..7210: mol_type = other DNA
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccaccatggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagttacccctatgacgtaccggattatgcatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 2 - DNA, 7282 bp, synthetic construct
  source 1..7282: mol_type = other DNA
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccaccatggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatagttctgtcccaagaagaactctgaagatgattcagccttctgcatctggatctggtggcggagggagttacccctatgacgtaccggattatgcatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 3 - DNA, 7243 bp, synthetic construct
  source 1..7243: mol_type = other DNA
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccaccatggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagttaaaatagttctgtcccaagaagaactctgaagatgattcagccttctgcatctggatcttgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 4 - DNA, 7240 bp, synthetic construct
  source 1..7240: mol_type = other DNA
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccaccatggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatagttctgtcccaagaagaactctgaagatgattcagccttctgcatctggatcttgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 5 - DNA, 7243 bp, synthetic construct
  source 1..7243: mol_type = other DNA
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccaccatggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagttaaaatagttctgtcccaagaagaactctgaagatgattcagccttctgcatctggatcttgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 6 - DNA, 7240 bp, synthetic construct
  source 1..7240: mol_type = other DNA
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccaccatggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatagttctgtcccaagaagaactctgaagatgattcagccttctgcatctggatcttgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 7 - DNA, 7240 bp, synthetic construct
  source 1..7240: mol_type = other DNA
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccaccatggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatagttctgtcccaagaagaactgcaaagatgattcagccttctgcatctggatcttgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 8 - DNA, 7240 bp, synthetic construct
  source 1..7240: mol_type = other DNA
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccaccatggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatagttctgtcccaagaagaactctgcgtatgattcagccttctgcatctggatcttgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 9 - DNA, 7240 bp, synthetic construct
  source 1..7240: mol_type = other DNA
gggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccaccatggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatagttctgtcccaagaagaactgcaaagatgattcagccttctgcatctggatcttgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagct

SEQ ID NO: 10 - RNA, 20 nt, synthetic construct
  source 1..20: mol_type = other RNA
ggggccactagggacaggat

SEQ ID NO: 11 - RNA, 20 nt, synthetic construct
  source 1..20: mol_type = other RNA
gttggagcatctgagtccag

SEQ ID NO: 12 - RNA, 20 nt, synthetic construct
  source 1..20: mol_type = other RNA
aattatggggattactagga

SEQ ID NO: 13 - RNA, 20 nt, synthetic construct
  source 1..20: mol_type = other RNA
gagaatcaaaatcggtgaat

SEQ ID NO: 14 - RNA, 20 nt, synthetic construct
  source 1..20: mol_type = other RNA
aagttcctgtgatgtcaagc

SEQ ID NO: 15 - RNA, 20 nt, synthetic construct
  source 1..20: mol_type = other RNA
taaacccggccactttcagg

SEQ ID NO: 16 - DNA, 54 bp, synthetic construct
  source 1..54: mol_type = other DNA
acactctttccctacacgacgctcttccgatcttcagaactgtccttcaggttc

SEQ ID NO: 17 - DNA, 56 bp, synthetic construct
  source 1..56: mol_type = other DNA
gtgactggagttcagacgtgtgctcttccgatctcagggcattacaaaagagaaga

SEQ ID NO: 18 - DNA, 25 bp, synthetic construct
  source 1..25: mol_type = other DNA
ctgtagttgttcacctgccrcaaga

SEQ ID NO: 19 - DNA, 24 bp, synthetic construct
  source 1..24: mol_type = other DNA
aatcctgtgcaaacccagrgctag

SEQ ID NO: 20 - DNA, 28 bp, synthetic construct
  source 1..28: mol_type = other DNA
tggcctcttggttttacagatarcgaac

SEQ ID NO: 21 - DNA, 28 bp, synthetic construct
  source 1..28: mol_type = other DNA
gctcaggctttggacttaaaagrcaaga

SEQ ID NO: 22 - protein, 5 aa, Synthetic construct
GGGGS

SEQ ID NO: 23 - protein, 9 aa, synthetic construct
YPYDVPDYA

SEQ ID NO: 24 - protein, 1403 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSYPYDVPDYA

SEQ ID NO: 25 - protein, 1427 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSNSSVPRRTLKMIQPSASGSGGGGSYPYDVPDYA

SEQ ID NO: 26 - protein, 1394 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGS

SEQ ID NO: 27 - protein, 1413 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSNSSVPRRTLKMIQPSASGS

SEQ ID NO: 28 - protein, 1394 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGS

SEQ ID NO: 29 - protein, 1413 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSNSSVPRRTLKMIQPSASGS

SEQ ID NO: 30 - protein, 1413 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSNSSVPRRTAKMIQPSASGS

SEQ ID NO: 31 - protein, 1413 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSNSSVPRRTLRMIQPSASGS

SEQ ID NO: 32 - protein, 1413 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSNSSVPRRTAKMIQPSASGS

SEQ ID NO: 33 - protein, 1517 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSNPSMKQKQEEIKENIKNSSVPRRTLKMIQPSASGSLVGRENELSAGLSKRKHRNDHLTSTTSSPGVIVPESSENKNLGGVTQESFDLMIKENPSSQYWKEVAEKRRKALGGGGSYPYDVPDYA

SEQ ID NO: 34 - protein, 1467 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSNPSMKQKQEEIKENIKNSSVPRRTLKMIQPSASGSLVGRENELSAGLSKRKHRNDHLTSGGGGSYPYDVPDYA

SEQ ID NO: 35 - protein, 1447 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAKRTADGSEFESPKKKRKVEGGGGSNPSMKQKQEEIKENIKNSSVPRRTLKMIQPSASGSLVGRGGGGSYPYDVPDYA

SEQ ID NO: 36 - protein, 19 aa, synthetic construct
NSSVPRRTLKMIQPSASGS

SEQ ID NO: 37 - DNA, 4554 bp, synthetic construct
  source 1..4554: mol_type = other DNA
atggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatcccagtatgaagcagaaacaagaagaaatcaaagagaatataaagaatagttctgtcccaagaagaactctgaagatgattcagccttctgcatctggatctcttgttggaagagaaaatgagctgtccgcaggcttgtccaaaaggaaacatcggaatgaccacttaacatctacaacttccagccctggggttattgtcccagaatctagtgaaaataaaaatcttggaggagtcacccaggagtcatttgatcttatgattaaagaaaatccatcctctcagtattggaaggaagtggcagaaaaacggagaaaggcgctgggtggcggagggagttacccctatgacgtaccggattatgcatga

SEQ ID NO: 38 - DNA, 4404 bp, synthetic construct
  source 1..4404: mol_type = other DNA
atggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatcccagtatgaagcagaaacaagaagaaatcaaagagaatataaagaatagttctgtcccaagaagaactctgaagatgattcagccttctgcatctggatctcttgttggaagagaaaatgagctgtccgcaggcttgtccaaaaggaaacatcggaatgaccacttaacatctggtggcggagggagttacccctatgacgtaccggattatgcatga

SEQ ID NO: 39 - DNA, 4344 bp, synthetic construct
  source 1..4344: mol_type = other DNA
atggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatcccagtatgaagcagaaacaagaagaaatcaaagagaatataaagaatagttctgtcccaagaagaactctgaagatgattcagccttctgcatctggatctcttgttggaagaggtggcggagggagttacccctatgacgtaccggattatgcatga

SEQ ID NO: 40 - DNA, 4512 bp, synthetic construct
  source 1..4512: mol_type = other DNA
atggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatcccagtatgaagcagaaacaagaagaaatcaaagagaatataaagaatagttctgtcccaagaagaactctgaagatgattcagccttctgcatctggatctcttgttggaagagaaaatgagctgtccgcaggcttgtccaaaaggaaacatcggaatgaccacttaacatctacaacttccagccctggggttattgtcccagaatctagtgaaaataaaaatcttggaggagtcacccaggagtcatttgatcttatgattaaagaaaatccatcctctcagtattggaaggaagtggcagaaaaacggagaaaggcgctgtga

SEQ ID NO: 41 - DNA, 4362 bp, synthetic construct
  source 1..4362: mol_type = other DNA
atggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatcccagtatgaagcagaaacaagaagaaatcaaagagaatataaagaatagttctgtcccaagaagaactctgaagatgattcagccttctgcatctggatctcttgttggaagagaaaatgagctgtccgcaggcttgtccaaaaggaaacatcggaatgaccacttaacatcttga

SEQ ID NO: 42 - DNA, 4302 bp, synthetic construct
  source 1..4302: mol_type = other DNA
atggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatgccaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgacgccgctaaaagaaccgccgatggcagcgagtttgagagccccaagaagaagcggaaggtcgagggtggcggagggagtaatcccagtatgaagcagaaacaagaagaaatcaaagagaatataaagaatagttctgtcccaagaagaactctgaagatgattcagccttctgcatctggatctcttgttggaagatga